(19) United States
(12) Patent Application Publication
(10) Pub. No.: US 2026/0007708 A1
(43) Pub. Date: Jan. 8, 2026

(54) FUNCTIONAL NATURAL COMPOSITION FOR PROMOTING GROWTH

(71) Applicant: FAMENITY CO., LTD., Uiwang-si (KR)

(72) Inventors: Tae Wan KIM, Gwacheon-si (KR); Tae Joon KIM, Gwacheon-si (KR)

(21) Appl. No.: 19/257,943

(22) Filed: Jul. 2, 2025

(30) Foreign Application Priority Data
Jul. 3, 2024 (KR) 10-2024-0087251

(51) Int. Cl. A61K 36/282; A61K 36/53; A61P 19/00

(52) U.S. Cl. CPC A61K 36/282; A61K 36/53; A61P 19/00; A61K 2236/331

(57) ABSTRACT

An embodiment relates to a composition for promoting height growth comprising an Artemisia dubia Wall. extract as an active ingredient, and may provide a composition for promoting height growth comprising an Artemisia dubia Wall. extract as an active ingredient, which has excellent effects of increasing body weight, increasing the tibial growth plate, increasing the femoral growth plate, and increasing the expression of a blood growth factor.

### CROSS REFERENCE TO RELATED APPLICATION

The present application claims priority to Korean Patent Application No. 10-2024-0087251, filed Jul. 3, 2024, the entire contents of which are incorporated here for all purposes by this reference.

### BACKGROUND

### 1. Technical Field

The present disclosure relates to a composition for promoting height growth comprising an Artemisia dubia Wall. extract as an active ingredient, and relates to a composition which, by comprising an Artemisia dubia Wall. extract as an active ingredient, exhibits the effects of increasing body weight, increasing the tibial growth plate, increasing the femoral growth plate, and increasing the expression of a blood growth factor and has excellent functionality.

### 2. Related Art

In our society, the developmental status of infants, children, and adolescents tends to be significantly improved due to improved nutritional status and changed eating habits as a result of recent economic growth. In general, the meaning of growth is often limited to an increase in height, but in medical terms, growth refers to an increase in morphological and anatomical size and function, which involves the enlargement and strengthening of the skeleton and muscles. Such growth is achieved by the action of hormones.

This has led to an increase in interest in height growth factors. Growth is defined in a broad sense to include not only an increase in height but also an increase in the size and function of each organ in the body, but is defined in a narrow sense to mean an increase in height. This increase in height is achieved by skeletal metabolism, including synthesis of cartilage tissue, an increase in skeletal length, and extensive proliferation of skeletal tissue, which are mediated by several factors, including nutritional and growth hormones. Bone tissue is a specially differentiated form of connective tissue, which is composed of various types of cells such as mesenchymal cells, chondrocytes, osteoblasts, osteoclasts, and bone marrow cells. There is a balance between the amount of bone resorption by osteoclasts and the amount of bone formation by osteoblasts. During the growth period, the ability of osteoblasts to form bone reaches its peak, and the amount of bone formation far exceeds the amount of bone resorption, resulting in bone growth.

Bone growth is achieved by the interaction between osteoclasts and osteoblasts. When bone is resorbed by osteoclasts, osteoblasts can absorb calcium and phosphorus, resulting in bone growth. When the activity of osteoblasts is higher than that of osteoclasts, bone growth occurs, and in the opposite case, bone destruction occurs, which can cause osteoporosis. The period when the activity of osteoblasts is higher than that of osteoclasts is the period when growth hormones are secreted, which is mainly the growth period of children or adolescents.

Human growth mainly takes place during the period up to puberty, and if proper nutrition is not consumed during this period, growth will not take place properly. Unlike adults, the growth plates of children's bones during the growth period are open, and the area of the growth plate increases through the intake of various nutrients and proper exercise during this period, resulting in an increase in overall height. Since this growth plate closes around 16 years of age for boys and around 14 years of age for girls, children in the growth period should receive sufficient nutrition during this period to promote the growth of the growth plate.

Methods for height growth include administration of growth hormone preparations, Ilizarov surgery, and taking health supplements. However, the growth hormone therapy has been found to have problems such as excessive duration and cost, cancer occurrence, and interference with other growth factors. Ilizarov surgery, a surgery that cuts and lengthens leg bones, is difficult to use for the general public, in terms of the patient's pain and cost. In addition, most health supplements for growth promotion are not scientifically proven.

That is, the administration of growth hormones may increase insulin resistance, increase the risk of type 2 diabetes, and cause various side effects such as increased blood sugar levels and the possibility of developing type 2 diabetes. In addition, the Ilizarov surgery is a surgical method that is mainly used when the lengths of both lower limbs are asymmetrical, but when the surgery is performed on a normal person who is short, it may cause serious side effects such as difficulty in walking normally.

In recent studies, a method of using natural products has attracted attention as a method of safely inducing growth while avoiding the side effects of growth hormone administration and the side effects of surgical procedures. Specifically, a method capable of stimulating the secretion of growth hormones by natural intake of natural products, ultimately inducing height growth, has been studied and accordingly, the search for natural products has been actively conducted.

Accordingly, the inventors of the present disclosure have conducted studies to provide a composition comprising an Artemisia dubia Wall. extract and a Thymus vulgaris L. extract as active ingredients, which is capable of inducing height growth while minimizing side effects, thereby completing the present disclosure.

### PRIOR ART DOCUMENTS

### Patent Documents

(Patent Document 1) KR 10-2572487 B1
(Patent Document 2) KR 10-2230955 B1

### SUMMARY

An object of the present disclosure is to provide a composition for promoting height growth comprising an Artemisia dubia Wall. extract as an active ingredient.

Another object of the present disclosure is to provide a composition that has an excellent activity of expressing a blood growth factor, by comprising an Artemisia dubia Wall. extract as an active ingredient.

Still another object of the present disclosure is to provide a food composition or a pharmaceutical composition comprising a composition for promoting height growth comprising an Artemisia dubia Wall. extract as an active ingredient.

### DETAILED DESCRIPTION

A composition for promoting height growth according to one embodiment of the present disclosure comprises an Artemisia dubia Wall. extract as an active ingredient.

The extract is obtained by extraction with an extraction solvent selected from the group consisting of water, a C₁-C₆ lower alcohol, and a mixture thereof.

The composition exhibits an effect of increasing the expression of a blood growth factor.

The blood growth factor is IGF-1 gene.

The composition further comprises a Thymus vulgaris L. extract.

The composition exhibits the effect of increasing body weight, increasing the tibial growth plate, or increasing the femoral growth plate.

A food composition for promoting height growth according to another embodiment of the present disclosure comprises the composition for promoting height growth.

A pharmaceutical composition for promoting height growth according to still another embodiment of the present disclosure comprises the composition for promoting height growth.

Hereinafter, the present disclosure will be described in more detail.

All technical terms used in the present disclosure, unless otherwise defined, have the following definitions and have the same meaning as commonly understood by those skilled in the art to which the present disclosure pertains. In addition, although preferred methods or samples are described herein, those similar or equivalent thereto are also included in the scope of the present disclosure.

In the specification of the present disclosure, plants are not limited in type, and those from any source, such as those cultivated or those purchased commercially, may be used without limitation.

In the specification of the present disclosure, the extract may be obtained by extraction using a method known in the art, and the method is not particularly limited. Alternatively, a commercially available extract may be used.

The plant extract may be obtained using any part of the plant, and the extraction part is not limited. The preparation of the plant extract is not limited by the type of plant, and the plant includes all those that have undergone processing processes such as drying. For example, the plant may be the whole plant, root, stem, leaf, fruit, flower, shoot, branch, bark, sap, principal bud, and/or seed of the aforementioned plant.

The natural extract used in the present disclosure may be prepared in a powder form by additional processes such as reduced-pressure distillation and freeze drying or spray drying.

As used herein, the term “prevention” means any action of inhibiting or delaying the progression of a disease or disorder, to which the term applies, or one or more symptoms of the disease or disorder, by administering the composition of the present disclosure.

As used herein, the term “amelioration” means any action of ameliorating or beneficially changing a disease or disorder, to which the term applies, or one or more symptoms of the disease or disorder, by administering the composition of the present disclosure.

As used herein, the term “treatment”, unless otherwise stated, means reversing, alleviating, inhibiting the progression of, or preventing a disease or disorder, to which the term applies, or one or more symptoms of the disease or disorder. The term “treatment” as used herein refers to the action of treating when “treating” is defined as above.

As used herein, the term “active ingredient” means an ingredient that exhibits a desired activity alone or may exhibit activity together with a carrier that has no activity by itself.

As used herein, the term “extract” has the meaning commonly known in the art as a crude extract as described above, but in a broad sense also includes a fraction obtained by further fractionating the extract. That is, the plant extract includes not only one obtained using the above-described extraction solvent, but also one obtained by additionally applying a purification process thereto. For example, the plant extract of the present disclosure also includes a fraction obtained by passing the extract through an ultrafiltration membrane having a predetermined molecular weight cut-off value, or a fraction obtained by additionally performing various purification methods, such as separation by various chromatography methods (designed for separation according to size, charge, hydrophobicity, or affinity).

In addition, the term “extract” includes the extract itself and an extract in any form that may be prepared using the extract, such as a dilution or concentrate of the extract, a dried product obtained by drying the extract, a crude or purified product of the extract, a fermented product of the extract, or a mixture thereof. In addition, the term “extract” includes a juice obtained by directly pressing or crushing a plant, followed by filtration. The plant may be extracted as it is or may be extracted after being processed.

The term “processing” refers to a pharmaceutical technology that changes the original properties of a medicinal herb by processing the medicinal herb based on oriental medicine theory. Examples of the processing include a stir-frying method of frying a medicinal herb, a baking method of baking a medicinal herb with a certain amount of liquid auxiliary ingredients so that the auxiliary ingredients penetrate into the medicinal herb tissue, and a steaming method of adding and mixing liquid auxiliary ingredients according to the processing regulations for each medicinal herb, and steaming them in an appropriate container by heating, or steaming them until they reach a certain degree, followed by drying. In addition, the term “extract” may mean a product obtained from a strain itself, a disrupted product of the strain, a culture thereof, a dead body thereof, or a mixture thereof by extraction with an extraction solvent.

As used herein, the term “fraction” means a product obtained by performing fractionation to separate a specific component or a specific group of components from a mixture containing various components.

The terminology used herein is for the purpose of describing embodiments only and is not intended to limit the present disclosure. In the present specification, singular forms also include plural forms unless the context clearly indicates otherwise. As used herein, “comprises” and/or “comprising” are/is intended to denote the existence of one or more stated components, steps, operations, and/or elements, but do/does not exclude the probability of existence or addition of one or more other components, steps, operations, and/or elements.

Specifically, among the terms used in the present specification, “comprising . . . as an active ingredient” means comprising an amount sufficient to achieve the efficacy or activity of the following natural extracts.

As used herein, the term “about” refers to a quantity, level, value, number, frequency, percentage, dimension, size, amount, weight, or length that varies by as much as about 30%, about 25%, about 20%, about 15%, about 10%, about 9%, about 8%, about 7%, about 6%, about 5%, about 4%, about 3%, about 2%, or about 1% to a reference quantity, level, value, number, frequency, percentage, dimension, size, amount, weight, or length.

The food composition may include a health functional food. As used herein, the term “health functional food” refers to a group of foods to which value has been added to food using physical, biochemical, or bioengineering techniques so that the function of the food is exerted and exhibited for a predetermined purpose, or a food designed and processed so that the bioregulatory functions of the food composition, such as regulation of the biological defense rhythm, prevention of disease, and recovery from disease are sufficiently exhibited in vivo. The health functional food has an active health maintenance or promotion effect compared to general foods, and the term “health supplement food” refers to a food for the purpose of health supplement. In some cases, the terms “functional food”, “health food”, and “health supplement food” are used interchangeably.

As used herein, the term “pharmaceutical composition” may be used as a concept including the meaning of “quasi-drug” or “medicine”.

The pharmaceutical composition of the present disclosure may be administered in a pharmaceutically effective amount. As used herein, the term “pharmaceutically effective amount” refers to an amount sufficient to treat or prevent a disease at a reasonable benefit/risk ratio applicable to any medical treatment or prevention. The effective dose level of the pharmaceutical composition may be determined depending on factors, including the severity of the disease, the activity of the drug, the patient's age, body weight, health, and gender, the patient's sensitivity to the drug, the administration time, administration route, and excretion rate of the composition of the present disclosure, the duration of treatment, and drugs that are used in combination or concurrently with the composition of the present disclosure, as well as other factors well known in the medical field. The pharmaceutical composition of the present disclosure may be administered alone or in combination with known immunotherapeutic agents. It is important to administer the pharmaceutical composition in the minimum amount that can exhibit the maximum effect without causing side effects, in view of all the above-described factors.

In the present disclosure, the “quasi-drug” may further comprise a pharmaceutically acceptable carrier, excipient or diluent as needed, in addition to comprising the plant extract as an active ingredient, The pharmaceutically acceptable carrier, excipient or diluent is not limited as long as it does not impair the effect of the present disclosure, and examples thereof include, but are not limited to, fillers, extenders, binders, wetting agents, disintegrating agents, surfactants, lubricants, sweeteners, fragrances, or preservatives.

A composition for promoting height growth according to one embodiment of the present disclosure comprises an Artemisia dubia Wall. extract as an active ingredient.

Preferably, the composition comprises an extract of the leaf of Artemisia dubia Wall. as an active ingredient, without being limited thereto.

Artemisia dubia Wall. is a vascular plant belonging to the family Asteraceae of the order Asterales. The rhizome extends, and the stem is erect, 1.5 to 2 m in height, and covered with cobweb-like hairs. The leaves are alternate and deeply divided into bipinnate leaves. The leaves attached to the middle of the stem are about 11 to 14 cm in length and about 8 mm in width. The leaflets are lanceolate, 3 to 4 cm in length and 0.5 to 1 cm in width, and the surface thereof is covered with cobweb-like hairs and has white spots. The flowers bloom in September to October, and the flower heads are borne in narrow panicles, and 3 mm in width. The involucre is tubular or bell-shaped, and densely covered with cobweb-like hairs, and the involucre segments are arranged in 3 to 4 rows. The fruit is an achene and is oval, 1 mm long, and hairless. Artemisia dubia Wall. is a perennial herb that grows in open grasslands or on mountain slopes. It is distinguished from Artemisia montana (Nakai) Pamp. of the genus Artemisia in that the leaves have white spots on the surface, are deeply divided into bipinnate leaves, and the leaflets are lanceolate and smooth. Young leaves are edible, and the whole plant is used medicinally. Artemisia dubia Wall. grows throughout Korea, and is distributed in Taiwan, India, Japan, China, and the like.

The Artemisia dubia Wall. extract is obtained by extraction with an extraction solvent selected from the group consisting of water, a C₁ to C₆ lower alcohol, and a mixture thereof.

Methods for preparing the above extract include conventional extraction methods known in the art, such as a solvent extraction method, a hot-water extraction method, a filtration method, an ultrasonic extraction method, a leaching method, and a reflux extraction method, and these methods may be performed alone or in combination of two or more thereof. In the present disclosure, the type of extraction solvent that is used for extraction is not particularly limited, and any solvent known in the art may be used.

Non-limiting examples of the extraction solvent include water; lower alcohols having 1 to 6 carbon atoms, such as methanol, ethanol, propyl alcohol, and butyl alcohol; polyhydric alcohols, such as glycerin, butylene glycol, and propylene glycol; hydrocarbon-based solvents, such as methyl acetate, ethyl acetate, acetone, benzene, hexane, diethyl ether, and dichloromethane; and mixtures thereof. Specifically, water, a lower alcohol, 1,3-butylene glycol, and ethyl acetate may be used alone or in combination of two or more thereof. In this case, when two or more types of solvents are used in combination, the mixing ratio between the solvents is not particularly limited.

Specifically, the Artemisia dubia Wall. extract may be an extract obtained from Artemisia dubia Wall., from which foreign substances have been removed by washing and drying, by extraction with water, an alcohol having 1 to 6 carbon atoms, or a mixed solvent thereof, or may be an extract obtained by sequentially applying the above solvents to Artemisia dubia Wall.

For extraction, Artemisia dubia Wall. may be left in the extraction solvent for leaching for 1 to 72 hours, more specifically for 24 to 48 hours.

In the present disclosure, the extraction may be performed using a solvent in an amount of 1 to 100 times, specifically 1 to 50 times, more specifically 2 to 20 times the weight of the dried plant, at an extraction temperature of 10 to 80° C. for an extraction time of 2 hours to 30 days, specifically 12 hours to 18 days, and may include a process of performing extraction on the dried and crushed plant material once or 2 to 5 consecutive times to obtain a liquid crude extract.

The method for preparing the extract may further comprise a step of fractionating the extract, obtained by extraction with the organic solvent, using an organic solvent, and the method may be a conventional extraction method known in the art, such as an ultrasonic extraction method, a leaching method, or a reflux extraction method.

The reflux extraction method is performed on 10 to 30 g, per 100 ml of water or an alcohol having 1 to 6 carbon atoms, of a crushed natural product for a reflux time of 1 to 3 hours using water and a 50 to 100% alcohol having 1 to 6 carbon atoms. More specifically, the reflux extraction method is performed on 10 to 20 g, per 100 mL of water or an alcohol having 1 to 6 carbon atoms, of a crushed natural product for a reflux time of 1 to 2 hours using water and a 70 to 90% alcohol having 1 to 4 carbon atoms. In addition, the extraction is repeated 1 to 5 times.

The leaching method is performed at 15 to 30° C. for 24 to 72 hours using water or a 50 to 100% alcohol having 1 to 6 carbon atoms as an extraction solvent. More specifically, it is performed at 20 to 25° C. for 30 to 54 hours using water or a 70 to 80% alcohol having 1 to 6 carbon atoms as an extraction solvent.

The ultrasonic extraction method is performed at 30 to 50° C. for 0.5 to 2.5 hours using water or a 50 to 100% alcohol having 1 to 6 carbon atoms as an extraction solvent. Specifically, it is performed at 40 to 50° C. for 1 to 2.5 hours using water or a 70 to 80% alcohol having 1 to 6 carbon atoms as an extraction solvent.

The extraction solvent may be used in an amount of 2 to 50 times, more specifically 2 to times the weight of Artemisia dubia Wall. For extraction, Artemisia dubia Wall. may be left in the extraction solvent for 1 to 72 hours, more specifically 24 to 48 hours.

After extraction, the extract may be fractionated using a fresh fractionation solvent. The fractionation solvent that is used for fractionation is any one or more selected from the group consisting of water, hexane, butanol, ethyl acetate, methylene chloride, and mixtures thereof, and is preferably ethyl acetate or methylene chloride. After obtaining the extract or the fraction, a method such as concentration or freeze-drying may be additionally performed thereon.

The composition for promoting height growth according to one embodiment of the present disclosure is characterized in that it can help height growth. Thus, the composition may also be used for the purpose of preventing or ameliorating growth disorder.

In the present disclosure, the term “growth disorder” means normal variant short stature or short stature secondary to a disease. The normal variant short stature may be familial short stature, constitutional growth retardation, or idiopathic short stature in a narrow sense. In addition, short stature secondary to a disease may be primary growth disorder (intrinsic disorder) or secondary growth disorder (extrinsic disorder). Examples of the primary growth disorders include short stature caused by osteochondrodysplasia, chromosomal abnormalities (Down syndrome or Turner syndrome), small for gestational age (intrauterine growth retardation), short stature caused by Prader-Willi syndrome, short stature caused by Russell-Silver syndrome, and short stature caused by Noonan syndrome, and examples of the secondary growth disorder include short stature caused by chronic systemic diseases, short stature caused by growth hormone deficiency, short stature caused by hypothyroidism, short stature caused by Cushing syndrome, and psychosocial dwarfism.

The composition for promoting height growth according to one embodiment of the present disclosure is characterized by exhibiting an effect of increasing growth factor expression, wherein the growth factor may be at least one selected from the group consisting of transforming growth factor-beta1 (TGF-β1), epidermal growth factor (EGF), insulin-like growth factor-1 (IGF-1), bone morphogenic protein-2 (BMP-2), platelet-derived growth factor (PDGF), osteogenic protein-1 (OP-1), acidic fibroblast growth factor (aFGF), and basic fibroblast growth factor (bFGF).

Growth hormone is secreted in a pulsatile manner from the anterior pituitary gland by the interaction of two peptide hormones of the hypothalamus: growth hormone-releasing hormone (GHRH), which promotes growth hormone secretion, and somatostatin, which inhibits growth hormone secretion. Growth hormone secreted into the blood mainly promotes the production of insulin-like growth factor-I (IGF-I) in the liver, wherein IGF-I is involved in the differentiation and growth of bone cells. IGF-I binds to insulin-like growth factor binding proteins (IGFBPs) in the blood, wherein IGFBPs play a role in promoting or inhibiting the biological action of IGF-I, and among these, IGFBP-3 in particular is closely related to the concentration of growth hormone in the blood.

In addition to its growth effect, growth hormone increases blood sugar levels by inducing reversible insulin resistance through metabolic action and has a lipolytic effect. Thus, obesity occurs when growth hormone is deficient. Growth hormone also increases lean body mass and bone density in vivo.

Specifically, the growth factor may be at least one selected from the group consisting of insulin-like growth factor-1 (IGF-1) and bone morphogenic protein-2 (BMP-2).

More specifically, the growth factor is blood insulin-like growth factor-1 (IGF-1).

In order to evaluate the height growth promoting effect of the composition for promoting height growth according to one embodiment of the present disclosure, insulin-like growth factor-1 (IGF-1) and IGFBP-3 (insulin-like growth factor binding protein-3 (IGFBP-3) were checked.

Preferably, the composition for promoting height growth according to the present disclosure may further comprise a natural extract, in addition to the Artemisia dubia Wall. extract.

The natural extract may be selected from the group consisting of a Thymus vulgaris L. extract, a Morus alba extract, a Pueraria lobata extract, a Cichorium intybus extract, and mixtures thereof.

Specifically, the natural extract may be selected from the group consisting of a Thymus vulgaris L. leaf extract, a Morus alba leaf extract, a Pueraria lobata leaf extract, a Cichorium intybus root extract, and mixtures thereof.

More preferably, the natural extract may be a Thymus vulgaris L. leaf extract.

Thymus vulgaris L. is a deciduous semi-shrub belonging to the family Lamiaceae of the order Lamiales of the class Magnoliopsida, and includes Thymus magnus Nakai that grows in rocky areas along the Korean coast, and Thymus quinquecostatus for. albus Y. N. Lee with white flowers. Thymus vulgaris L. is used as a beverage, a disinfectant, a cosmetic ingredient, or medicine.

Preferably, the composition for promoting height growth according to the present disclosure may comprise, based on 100 parts by weight of a Artemisia dubia Wall. leaf extract, 10 to 100 parts by weight of a Thymus vulgaris L. leaf extract, more preferably 10 to 75 parts by weight of a Thymus vulgaris L. leaf extract, even more preferably 25 to 75 parts by weight of a Thymus vulgaris L. leaf extract.

When the composition for promoting height growth according to the present disclosure further comprises, in addition to the Artemisia dubia Wall. extract, a Thymus vulgaris L. extract, it may exhibit excellent effects of increasing body weight, increasing the tibial growth plate, increasing the femoral growth plate, and increasing the expression of a blood growth factor, compared to the composition comprising only the Artemisia dubia Wall. extract.

The composition may further comprise, in addition to a mixture of the Artemisia dubia Wall. extract and the Thymus vulgaris L. extract, γ-oryzanol. γ-Oryzanol is an antioxidant derived from rice bran and has an excellent reactive oxygen species (ROS) removal effect. In addition, γ-oryzanol has been reported to have an effect of balancing the endocrine system by acting on the regulation of hypothalamus and pituitary gland functions. In particular, γ-oryzanol may create an environment suitable for growth hormone secretion by alleviating stress and stabilizing the autonomic nervous system.

Accordingly, the composition according to the present disclosure may comprise, based on 100 parts by weight of the Artemisia dubia Wall. extract, 10 to 100 parts by weight of a Thymus vulgaris L. leaf extract and 1 part by weight of γ-oryzanol, more preferably 10 to 75 parts by weight of a Thymus vulgaris L. leaf extract and 1 part by weight of γ-oryzanol, even more preferably, 25 to 75 parts by weight of a Thymus vulgaris L. leaf extract and 1 part by weight of γ-oryzanol.

The composition according to the present disclosure may further comprise, in addition to a mixture of the Artemisia dubia Wall. extract and the Thymus vulgaris L. extract, a natural extract.

The natural extract may be selected from the group consisting of a Morus alba leaf extract, a Pueraria lobata leaf extract, a Cichorium intybus root extract, and mixtures thereof.

Specifically, the natural extract may be selected from the group consisting of a Morus alba leaf extract, a Pueraria lobata leaf extract, a Cichorium intybus root extract, and mixtures thereof.

Preferably, the composition according to the present disclosure comprises, based on 100 parts by weight of a mixture of the Artemisia dubia Wall. extract and the Thymus vulgaris L. extract, 5 to 25 parts by weight of a Morus alba leaf extract, 5 to 25 parts by weight of a Pueraria lobata leaf extract, and 5 to 25 parts by weight of a Cichorium intybus root extract, more preferably to 20 parts by weight of a Morus alba leaf extract, 10 to 20 parts by weight of a Pueraria lobata leaf extract, and 10 to 20 parts by weight of a Cichorium intybus root extract.

It was confirmed that, when the composition further comprises, in addition to a mixture of the Artemisia dubia Wall. extract and the Thymus vulgaris L. extract, a Morus alba extract, a Pueraria lobata extract, and a Cichorium intybus extract, it exhibited excellent effects of increasing body weight, increasing the tibial growth plate, increasing the femoral growth plate, and increasing the expression of a blood growth factor, compared to the composition comprising only the mixture of the Artemisia dubia Wall. extract and the Thymus vulgaris L. extract.

A food composition according to another embodiment of the present disclosure may comprise the above-described composition.

When the plant extract of the present disclosure is provided as a food composition, the food composition may contain a food-acceptable food supplement additive, in addition to the active ingredient.

That is, the food composition in the present disclosure is not particularly limited in any other ingredients, except that it contains the natural extracts presented in the present disclosure. The food composition may contain various herbal extracts, food supplement additives, or natural carbohydrates as additional ingredients, like conventional foods.

The food composition according to the present disclosure may be provided in the form of powder, granules, tablets, capsules, syrups or beverages, may be used together with other foods or food additives, and may be used appropriately according to a conventional method. The content of the active ingredient may be appropriately determined depending on the purpose of use, for example, prevention, health care, or therapeutic treatment.

As a specific example, processed foods capable of enhancing the effect of promoting height growth may be manufactured using the food composition. Such processed foods include, for example, confectionery, beverages, alcoholic beverages, fermented foods, canned foods, processed milk foods, processed meat foods, noodles, etc. Confectionery includes biscuits, pies, cakes, bread, candy, jellies, gums, cereals, etc. Beverages include drinking water, carbonated beverages, functional sports drinks, juices (e.g., apple, pear, grape, aloe, tangerine, peach, carrot, or tomato juice, etc.), shikhye (a traditional Korean beverage made with rice), etc. Alcoholic beverages include refined rice wine, whiskey, soju, beer, whiskey, fruit wine, etc. Fermented foods include soy sauce, soybean paste, and red pepper paste. Canned foods include canned seafood (e.g., canned tuna, mackerel, saury pike, and conch), canned livestock products (e.g., canned beef, pork, chicken, and turkey), and canned agricultural products (e.g., canned corn, peaches, and pineapples). Processed milk foods include cheese, butter, yogurt, etc. Processed meat foods include pork cutlet, beef cutlet, chicken cutlet, sausage, sweet and sour pork, nuggets, and marinated grilled beef. Noodles include sealed packaged raw noodles. In addition, the composition may be used in retort foods, soups, etc.

The food supplement additives refer to components that may be added to food for preservation purposes. The food supplement additives are added to manufacture a health function food of each formulation and may be appropriately selected and used by those skilled in the art. Examples of the food supplement additives include, but are not limited to, various nutrients, vitamins, minerals (electrolytes), flavoring agents such as synthetic flavoring agents and natural flavoring agents, colorants and fillers, pectic acid and salts thereof, alginic acid and salts thereof, organic acids, protective colloidal thickeners, pH regulators, stabilizers, preservatives, glycerin, alcohols, carbonating agents used in carbonated beverages, and the like.

Specifically, the health functional food is a food prepared by adding the plant extract to food materials such as beverages, teas, spices, gum, and confectionery, or a food prepared by encapsulating, powdering, or suspending the plant extract. The health functional food means a food that has a specific health effect when consumed. However, unlike general drugs, the health functional food uses food as a raw material, and thus has the advantage of not having side effects that may occur when taking drugs for a long period of time.

The food may comprise food-acceptable food supplement additives, and may further comprise appropriate carriers, excipients and diluents that are commonly used in the manufacture of health functional foods.

Each of the above-described ingredients contained in the food composition according to the present disclosure may be contained in the food composition of the present disclosure within a range that does not exceed the maximum usage amount stipulated in the food safety standards of each country.

The composition may comprise additional ingredients that may be commonly used in food compositions to improve odor, taste, vision, etc. For example, the composition may include vitamins A, C, D, E, B1, B2, B6, B12, niacin, biotin, folate, pantothenic acid, etc. In addition, the composition may comprise minerals such as zinc (Zn), iron (Fe), calcium (Ca), chromium (Cr), magnesium (Mg), manganese (Mn), and copper (Cu). In addition, the composition may comprise amino acids such as lysine, tryptophan, cysteine, and valine. There is no limitation on the form that the health functional food of the present disclosure may take. The term “health functional food” may include all foods in the conventional sense, and may be used interchangeably with terms known in the art, such as functional food.

In addition, the health functional food of the present disclosure may be manufactured by mixing the composition of the present disclosure with other appropriate auxiliary ingredients and known additives that may be contained in food according to the selection of a person skilled in the art. Examples of foods to which the composition of the present disclosure may be added include dairy products, including meat, sausage, bread, chocolate, candy, snacks, confectionery, pizza, ramen, other noodles, gum, and ice cream, various soups, beverages, tea, drinks, alcoholic beverages, and vitamin complexes, and these foods may be manufactured by adding the food extract of the present disclosure as an active ingredient to juice, tea, jelly, and juice. These foods also include food that is used as feed for animals.

When the food formulation is a beverage, it may contain various flavoring agents or natural carbohydrates as additional ingredients, like a conventional beverage. The natural carbohydrates mentioned above are monosaccharides such as glucose and fructose, disaccharides such as maltose and sucrose, polysaccharides such as dextrin and cyclodextrin, and sugar alcohols such as xylitol, sorbitol and erythritol. As the flavoring agents, natural flavoring agents such as thaumatin and stevia extract, or synthetic flavoring agents such as saccharin and aspartame may be used. The proportion of the natural carbohydrates is generally about 0.01 to 0.04 g, preferably about 0.02 to 0.03 g, per 100 mL of the composition of the present disclosure.

In addition, the food formulation may contain various nutrients, vitamins, electrolytes, flavoring agents, colorants, pectic acid or salts thereof, alginic acid or salts thereof, organic acids, protective colloidal thickeners, pH adjusters, stabilizers, preservatives, glycerin, alcohols, carbonating agents used in carbonated drinks, etc. Additionally, the food formulation may contain fruit flesh for the preparation of natural fruit juices, fruit juice beverages and vegetable juices. These ingredients may be used alone or in combination. The proportion of these additives is not so critical, but is generally in the range of 0.01 to 0.1 parts by weight per 100 parts by weight of the composition of the present disclosure.

When the above plant extract is used as a food additive, the plant extract may be added alone or in combination with other foods or food ingredients, and may be used appropriately according to a conventional method. The amount of the active ingredient added may be appropriately determined depending on the intended use (prevention, health care, or therapeutic treatment). Generally, when manufacturing a food or a beverage, the composition of the present disclosure is added in an amount of 15 parts by weight or less, preferably 10 parts by weight or less, based on the total weight of the food or beverage. However, in the case of long-term intake for the purpose of health and hygiene or for the purpose of health care, the amount of the active ingredient may be smaller than the lower limit of the above range, and the active ingredient may be used in an amount larger than the upper limit of the above range as there is no problem in terms of safety.

The food composition of the present disclosure may contain conventional food additives, and suitability as the food additive is determined by the specifications and standards for the relevant item according to the general provisions and general test methods of the Food Additive Code approved by the Ministry of Food and Drug Safety, unless otherwise specified.

The items listed in the Food Additive Code include, for example, chemical compounds such as ketones, glycine, potassium citrate, nicotinic acid, and cinnamic acid; natural additives such as persimmon color, licorice extract, crystalline cellulose, high-molecular-weight pigment, and guar gum; and mixed preparations such as sodium L-glutamate preparations, alkaline agents for addition to noodles, preservative preparations, and tar color preparations.

The pharmaceutical composition of the present disclosure may be administered in a pharmaceutically effective amount. As used herein, the term “pharmaceutically effective amount” refers to an amount sufficient to treat or prevent a disease at a reasonable benefit/risk ratio applicable to any medical treatment or prevention. The effective dose level may be determined depending on factors, including the severity of the disease, the activity of the drug, the patient's age, body weight, health, and gender, the patient's sensitivity to the drug, the administration time, administration route, and excretion rate of the composition of the present disclosure, the duration of treatment, and drugs that are used in combination or concurrently with the composition of the present disclosure, as well as other factors well known in the medical field. The pharmaceutical composition of the present disclosure may be administered alone or in combination with known immunotherapeutic agents. It is important to administer the pharmaceutical composition in the minimum amount that can exhibit the maximum effect without causing side effects, in view of all the above-described factors.

The pharmaceutical composition according to another embodiment of the present disclosure may comprise the above-described composition.

The pharmaceutical composition may be in the form of a solution, suspension or emulsion in an oil or aqueous medium, or in the form of an extract, powder, granule, tablet or capsule.

In addition, the pharmaceutical composition of the present disclosure may further comprise a pharmaceutically acceptable carrier.

Pharmaceutically acceptable carriers include various ingredients such as buffers, sterile water for injection, normal saline or phosphate buffered saline, sucrose, histidine, salts, and polysorbates.

The pharmaceutical composition of the present disclosure may be administered orally or parenterally, and may be administered in the form of general pharmaceutical preparations, for example, in various oral and parenteral formulations for clinical administration. In the preparation of the formulation, the active ingredient is preferably mixed or diluted with a carrier or enclosed in a container-type carrier.

Examples of carriers that may be contained in the pharmaceutical composition of the present disclosure include, but are not limited to, lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methyl hydroxybenzoate, propyl hydroxy benzoate, talc, magnesium stearate, and mineral oil. For formulation, the pharmaceutical composition may be formulated using diluents or excipients such as commonly used fillers, extenders, binders, wetting agents, disintegrating agents, and surfactants.

Solid formulations for oral administration include tablets, pills, powders, granules, capsules, etc., and these solid formulations may be prepared by mixing the pharmaceutical composition of the present disclosure with one or more excipients, such as starch, calcium carbonate, sucrose or lactose, gelatin, etc.

In addition to simple excipients, lubricants such as magnesium stearate and talc are also used. Liquid formulations for oral administration include suspensions, solutions, emulsions, and syrups, and may contain, in addition to commonly used simple diluents such as water and liquid paraffin, various excipients such as wetting agents, sweeteners, flavoring agents, and preservatives.

Liquid formulations for oral administration include suspensions, solutions, emulsions, syrups, etc., and may contain, in addition to commonly used simple diluents such as water and liquid paraffin, various excipients such as wetting agents, sweeteners, flavoring agents, and preservatives.

Formulations for parenteral administration include sterile aqueous solutions, non-aqueous solutions, suspensions, emulsions, lyophilized formulations, and suppositories. Non-aqueous solvents and suspending agents include propylene glycol, polyethylene glycol, vegetable oils such as olive oil, and injectable esters such as ethyl oleate. Suppository bases include witepsol, macrogol, Tween 61, cacao butter, laurin fat, and glycerogelatin.

The effective amount of the plant extract contained in the composition of the present disclosure may vary depending on the form in which the composition is manufactured, the method by which the compound is applied to the skin, and the length of time the composition remains on the skin. For example, when the composition is manufactured in a pharmaceutical dosage form, the plant extract may be contained at a higher concentration than when the composition is manufactured as a cosmetic product that is routinely applied to the skin. Therefore, the daily dose may be 0.1 to 100 mg/kg, preferably 30 to 80 mg/kg, and more preferably 50 to 60 mg/kg as the amount of the plant extract, and the composition may be administered 1 to 6 times a day. The compositions of the present disclosure can be used alone or in combination with surgery, radiotherapy, hormone therapy, chemotherapy, and methods that use biological response modifiers.

In the present disclosure, the “quasi-drug” may further comprise, in addition to the plant extract as an active ingredient, a pharmaceutically acceptable carrier, excipient or diluent as needed. The pharmaceutically acceptable carrier, excipient or diluent is not limited as long as it does not impair the effect of the present disclosure, and examples thereof include, but are not limited to, fillers, extenders, binders, wetting agents, disintegrating agents, surfactants, lubricants, sweeteners, fragrances or preservatives.

The quasi-drug composition of the present disclosure may further comprise, as an active ingredient, at least one ingredient selected from the group consisting of caries preventive agents such as sodium fluoride, sodium fluorophosphate, amine fluoride, tin fluoride or xylitol; disinfectants such as chlorhexidine, cetylpyridium chloride or triclosan; herbal medicines such as bamboo salt or propolis; anti-inflammatory agents such as allantoin, aminocaproic acid, tranexamic acid, tocopherol acetate or methylsulfonylmethane; and enzymes such as dextranase, glucose oxidase, glucoamylase, α, β-amylase, lactoperoxidase, and lysozyme.

The quasi-drug may be, for example, a disinfectant, a shower foam, an ointment, a wet tissue, a coating agent, etc., and is preferably manufactured as a semi-solid formulation such as an ointment for external use, lotion, etc., without being limited thereto. The formulation method, dosage, method of use, components, etc. of the above-mentioned quasi-drug may be appropriately selected from conventional techniques known in the art.

In addition, the quasi-drug composition of the present disclosure may further comprise at least one substance selected from the group consisting of an abrasive, a humectant, a binder, a foaming agent, a sweetener, an anionic surfactant, a flavoring agent, and a pigment.

For use, the composition may be added alone to a quasi-product or mixed with or added to a functional food or functional ingredient. The specific formulation is defined as including all compositions and mixing ranges that may be selected and mixed by those skilled in the art.

The present disclosure relates to a composition for promoting height growth comprising an Artemisia dubia Wall. extract as an active ingredient, and may provide a composition for promoting height growth comprising an Artemisia dubia Wall. extract as an active ingredient, which has the effects of increasing body weight, increasing tibial growth plate, increasing femoral growth plate, and increasing the expression of a blood growth factor.

### DETAILED DESCRIPTION

Hereinafter, examples of the present disclosure will be described in detail so that those skilled in the art can easily carry out the present disclosure. However, the present disclosure may be embodied in various different forms and is not limited to the examples described herein.

### Preparation Example 1: Preparation of Natural Extracts

### Preparation of Artemisia dubia Wall. Extract

Artemisia dubia Wall. was washed, dried, and then ground. The ground material was added to distilled water, an extraction solvent, at a ratio of 1:10 (w:v), completely immersed therein, and then extracted three times under reflux at 70 to 80° C. for 2 hours each. The extract was filtered through Whatman No. 2 filter paper. The filtrate was concentrated under reduced pressure at 60° C., and then freeze-dried, thereby preparing a powder sample which was then homogenized and then used in the experiment.

### Preparation of Thymus vulgaris L. Extract

A Thymus vulgaris L. extract (TE) was prepared using the same method as the method for preparing the Artemisia dubia Wall. extract (AE).

### Preparation of Extract Mixtures

The Artemisia dubia Wall. extract (AE) and the Thymus vulgaris L. extract (TE) were mixed with each other at ratios within the weight ratio range shown in Table 1 below, thereby preparing extract mixtures.

TABLE 1

| | M1 | M2 | M3 | M4 | M5 | M6 |
|---|---|---|---|---|---|---|
| AE | 100 | 100 | 100 | 100 | 100 | 100 |
| TE | — | 10 | 25 | 50 | 75 | 100 |

(unit: parts by weight)

In addition, an extract mixture (M7) was additionally prepared by adding 1 part by weight of γ-oryzanol to M4.

### Example 1: Experiment on Promotion of Bone Growth by Artemisia dubia Wall. Extract

In order to confirm whether the plant extract prepared in Preparation Example 1 is effective in promoting bone growth by increasing the formation of a growth plate and the expression of IGF-1, an experiment was conducted to evaluate the effect of the plant extract on the formation and proliferation of a growth plate by administering the Artemisia dubia Wall. extract to young test animals in the growth period.

Eutropin PLUS Inj. (ingredient: human growth hormone, somatropin) was used as a positive control substance.

The data analysis and statistics system used was Prime (ver. 2.01) (Graphpad Software Inc., USA), and the experimental analysis device used was an ELISA reader (LAB System, USA).

Experimental animals, SD rats, were selected as they are a species that is widely used in growth promotion efficacy tests and have accumulated data, making it useful for comparison.

The sex, number, age, and body weight of the experimental animals are shown in Table 2 below.

TABLE 2

| | | Number of | Age | Body weight |
|---|---|---|---|---|
| | Sex | animals | (weeks) | (g) |
| When received | Male | 44 | 3 | 54.2 to 63.8 |
| When starting | Male | 40 | 4 | 112.2 to 134.3 |
| administration | | | | |

The housing environment of the experimental animals is shown in Table 3 below.

TABLE 3

| | |
|---|---|
| Housing box | Polycarbonate, 278 × 420 × 200 (mm, W × L × H) |
| Number of animals | Quarantine and acclimatization period: 2 or less |
| per box | Administration period: 2 |
| Temperature | 20 to 24° C. |
| Relative humidity | 70% |
| Number of | 10 to 15/hour |
| ventilations | |
| Lighting cycle | 12 hours (lights on: 7am and lights off: 7pm) |
| Luminous intensity | 600 lux or more |
| Management of | Replace the housing box twice a week and the water |
| housing equipment | supply bottle twice a week. |
| | Use the housing equipment after washing with |
| | disinfectant, rinsing with tap water, and complete |
| | drying. |

Solid feed for laboratory animals (+40 RMN, SAPE complete care competence, France) was placed in a feeder and allowed to be consumed ad libitum. Sterilized tap water was placed in a drinking bottle and allowed to be consumed ad libitum.

The number of animals was checked upon receipt, the observation of general symptoms and the measurement of body weight were performed, and test results provided by the animal supplier were stored as basic test data. General symptoms were observed once a day for all animals during the quarantine and acclimatization period.

No abnormal animals were found during the quarantine and acclimatization period.

During the acclimatization period, the tails of animals were marked with a red marker for individual identification, and individual identification cards for the quarantine and acclimatization period were attached to the housing boxes. During the experimental period, the tails of animals were marked with a red marker for group identification and with a blue marker for individual identification, and individual identification cards for the experimental period were attached to the housing boxes.

On the last day of the quarantine and acclimatization period (final quarantine day), the body weight was measured, general symptoms and body weight changes were checked to evaluate the health status of the animals, and animals without abnormal symptoms were used in the experiment.

After the final quarantine, based on the ranked weight measurement results, the animals were divided into 5 groups with 8 animals per group so that the average weight of each group was distributed as evenly as possible.

Grouping and administration are shown in Table 4 below.

TABLE 4

| | Administration | Administration | Number of |
|---|---|---|---|
| | dose | volume | animals |
| | (mg/kg) | (ml/kg) | (No.) |
| G1, normal control | — | 10 | 8 (101 to 108) |
| group (DW) | | | |
| G2, low-dose extract | 200 | 10 | 8 (201 to 208) |
| group | | | |
| G3, medium-dose | 600 | 10 | 8 (301 to 308) |
| extract group | | | |
| G4, high-dose extract | 1,800 | 10 | 8 (401 to 408) |
| group | | | |
| G5, positive control | 0.5 | 1 | 8 (501 to 508) |
| substance group | | | |

The administration volume of the extract was set at 10 ml/kg, and the administration volume for each individual was calculated based on the body weight measured close to the day of administration.

The administration volume of the positive control substance was set to 1 ml/kg, and the administration volume for each individual was calculated based on the body weight measured close to the day of administration.

Since the intended route for clinical application of the extract was oral, the oral route was selected for each extract group and the normal control group.

Since the route for clinical application of the positive control substance was subcutaneous, the subcutaneous route was selected for the positive control substance group.

The extract and an excipient were administered intragastrically once a day a total of 14 times for 14 days using a disposable syringe having an oral administration sonde attached thereto.

The positive control substance was injected subcutaneously once every two days a total of seven times over two weeks using a disposable insulin syringe.

Body weights were measured for all animals before the start of extract administration. Body weights were measured twice a week until the end of the experiment, and on the day of autopsy.

On the day of autopsy, the experimental animals were euthanized under CO₂ gas anesthesia, and the right hind limb tibia and femur were extracted.

The tibia and femur were measured for their length using a caliper immediately after extraction and photographed.

The extracted tibia and femur were fixed in 10% neutral formalin. After decalcification, they were embedded in paraffin and sectioned.

The sectioned tissues were stained with hematoxylin & eosin (H&E) and observed and imaged under a microscope, and the growth plate thickness was analyzed using ImageJ (Image) version 1.52p, National Institutes of Health, USA).

The results of measuring body weight, tibial growth plate thickness, femoral growth plate thickness, and expression of blood IGF-1 and IGFBP-3 genes in the experiment were presented as mean±S.E. and analyzed using SPSS (SPSS Version 21.0 Inc., U.S.A.). The normal control group (G1), each of the extract test groups (G2 to G4), and the positive control substance group (G5) were subjected to independent t-test to determine significance (significance level: one-sided 0.05).

Body weights were measured for all animals before administration of the test substance. Body weights were measured twice a week until the end of the experiment and measured on the day of necropsy.

The growth plate is the part of the limb bones where growth in length occurs, and is made up of cartilage cells at the ends of the bones. Growth hormone causes cell division in the cartilage cells of the growth plate, causing bone growth, and after puberty, cell division ceases and growth ends.

The tibial growth plate thickness and the femoral growth plate thickness were measured, and the results are shown in Table 5 below.

TABLE 5

| | Administration | Tibial growth | Femoral growth |
|---|---|---|---|
| | dose | plate thickness | plate thickness |
| | (mg/kg) | (μm) | (μm) |
| G1, normal control | — | 465.78 ± 14.59 | 507.68 ± 23.11 |
| group (DW) | | | |
| G2, low-dose | 200 | 486.77 ± 16.98 | 522.91 ± 31.29 |
| extract group | | | |
| G3, medium-dose | 600 | 501.61 ± 15.49 | 623.72 ± 40.91 |
| extract group | | | |
| G4, high-dose | 1,800 | 529.41 ± 17.31 | 638.17 ± 20.79 |
| extract group | | | |
| G5, positive control | 0.5 | 516.23 ± 10.37 | 603.79 ± 27.23 |
| substance group | | | |

Data presented mean ± S.E., N = 8
Significant difference from the normal control group by independent t-test:
* p < 0.05,
** p < 0.01
S.E.: Standard error
N: Number of animals

Referring to Table 5 above, the average thickness of the tibial growth plate in the normal control group was measured to be 465.31 μm. Meanwhile, the average thickness of the tibial growth plate in the low-dose extract group was 483.92 μm, and that in the medium-dose extract group was 500.18 μm, which did not show a significant difference from that in the normal control group. However, the average thickness of the tibial growth plate in the high-dose extract group was 527.04 μm, which was a statistically significant increase compared to that in the normal control group. The average thickness of the tibial growth plate in the positive control substance group was 515.34 μm, which was a statistically significant increase compared to that in the normal control group.

In addition, referring to Table 5 above, the above thickness of the femoral growth plate in the normal control group was measured to be 507.13 μm. Meanwhile, the average thickness of the femoral growth plate in the low-dose extract group was 519.91 μm, which was not significantly different from that in the normal control group. However, the average thickness of the femoral growth plate in the medium-dose extract group was 621.33 μm, and the average thickness of the femoral growth plate in the high-dose extract group was 634.87 μm, indicating that the femoral growth plate thickness in each of the medium-dose extract group and the high-dose extract group statistically significantly increased compared to that in the normal control group. The average thickness of the femoral growth plate in the positive control substance group was 601.79 μm, which was a statistically significantly increase compared to that in the normal control group. Blood total RNA was isolated using an RNA extraction kit (Cat No.: 74106, Qiagen, U.S.A.) according to the manufacturer's manual. The isolated total RNA was quantified by measuring the absorbance at 260 and 280 nm using a microplate reader (Infinite M200 pro, Tecan, Austria).

RT-PCR mixture was prepared using the RT-qPCR kit (TOPreal™ One-step RT-qPCR Mix, Cat No.: RT432M, Enzynomics, Republic of Korea). The RT-PCR mixture was placed into a 384-well plate which was then placed on the heat block of a real-time PCR machine (CFX384, BIORAD, CA, U.S.A.), and then RT-PCR reaction and analysis were performed.

The expression levels of blood IGF-1 and IGFBP-3 genes were measured, and the results are shown in Table 6 below.

TABLE 6

| | | IGF-1 gene | IGFBP-3 gene |
|---|---|---|---|
| | Administration | expression | expression |
| | dose (mg/kg) | level | level |
| G1, normal control | — | 0.99 ± 0.15 | 1.01 ± 0.02 |
| group (DW) | | | |
| G2, low-dose extract | 200 | 1.11 ± 0.11 | 0.97 ± 0.09 |
| group | | | |
| G3, medium-dose extract | 600 | 1.13 ± 0.12 | 0.99 ± 0.15 |
| group | | | |
| G4, high-dose extract | 1,800 | 1.36 ± 0.08 | 1.16 ± 0.12 |
| group | | | |
| G5, positive control | 0.5 | 1.34 ± 0.09 | 0.98 ± 0.08 |
| substance group | | | |

Data presented mean ± S.E., N = 8
Significant difference from the normal control group by independent t-test:
* p < 0.05,
** p < 0.01
S.E.: Standard error
N: Number of animals

The blood IGF-1 gene expression level statistically significantly increased in the high-dose extract group compared to the normal control group, and the IGFBP-3 gene expression level slightly increased in the high-dose extract group compared to the normal control group, but there was no statistically significant difference in the IGFBP-3 gene expression level between the normal control group and the test substance group.

Taking the above experimental results together, administration of the extract increased the length of the growth plate and increased the proportion of the proliferative region where cell division occurred. In addition, it was confirmed that the extract also increased the expression of IGF-1, which increases during growth.

Therefore, it can be seen that the composition for promoting bone growth comprising the extract according to the present disclosure helps bone growth by increasing the formation of a growth plate and the expression of IGF-1.

### Example 2: Experiment on Promotion of Bone Growth by Artemisia dubia Wall. Extract and Thymus vulgaris L. Extract

In order to confirm whether the natural extracts M2 to M7 prepared in Preparation Example 1 are effective in promoting bone growth by increasing the formation of the growth plate and the expression of IGF-1, an experiment was conducted using the same method as the method for the experiment on the promotion of bone growth by the Artemisia dubia Wall. extract, shown in Example 1. Specifically, in order to evaluate the experimental results for M2 to M7, which further contained the Thymus vulgaris L. extract (TE) in addition to the Artemisia dubia Wall. extract (AE), by a relative index value method, the experimental results for the group (M1) administered a high dose of the Artemisia dubia Wall. extract (AE) were set at index 3. In addition, for comparison with the effect of γ-oryzanol, γ-oryzanol (1,800 mg/kg, high-dose group) was administered as a control in the same manner as the high-dose group of the above-described experiment, and the result value was expressed as an index relative to that of M1. The experimental results are comprehensively shown in Table 7 below. In Table 7 below, the higher the index, the better the effect.

TABLE 7

| | M1 | M2 | M3 | M4 | M5 | M6 | M7 | Control |
|---|---|---|---|---|---|---|---|---|
| Body weight gain | 3 | 4 | 5 | 7 | 6 | 5 | 9 | 1 |
| Tibial growth plate | 3 | 4 | 6 | 8 | 7 | 6 | 9 | 1 |
| thickness | | | | | | | | |
| Femoral growth | 3 | 4 | 6 | 7 | 7 | 5 | 9 | 1 |
| plate thickness | | | | | | | | |
| IGF-1 expression | 3 | 5 | 7 | 9 | 8 | 6 | 9 | 1 |
| level | | | | | | | | |
| IGFBP-3 expression | 3 | 4 | 5 | 6 | 5 | 4 | 9 | 1 |
| level | | | | | | | | |

(unit: index)

Referring to Table 7 above, it can be seen that, in the experiment on bone growth promotion efficacy, the effect of M2 to M6 further comprising the Thymus vulgaris L. extract on body weight gain, tibial growth plate thickness, femoral growth plate thickness, and IGF-1 expression level were better than the effect of M1 containing only the Artemisia dubia Wall. extract. In addition, it could be confirmed that the effect of M2 to M6 further containing the Thymus vulgaris L. extract on IGFBP-3 expression level slightly increased compared to that of M1 containing only the Artemisia dubia Wall. extract.

Specifically, treatment with each of M2 to M6 further containing the Thymus vulgaris L. extract (TE) in addition to the Artemisia dubia Wall. extract (AE) exhibited an increased effect compared to treatment with M1 containing only the Artemisia dubia Wall. extract. Preferably, the mixtures M3 to M5 exhibited an increased effect.

In addition, it was confirmed that, in the case of M7 containing all the Artemisia dubia Wall. extract (AE), the Thymus vulgaris L. extract (TE) and γ-oryzanol, the effect was maximized due to the interaction between the components. As it was confirmed that the effect of the control was insignificant compared to that of M1, it can be said that the effect of M7 containing all the Artemisia dubia Wall. extract (AE), the Thymus vulgaris L. extract (TE) and γ-oryzanol was maximized compared to the effect of γ-oryzanol alone. Therefore, it could be seen that the composition according to the present disclosure was effective in promoting bone growth.

### Example 3: Preference Evaluation

Beverages containing the Artemisia dubia Wall. extract prepared in Preparation Example 1 were manufactured and tasted by 30 male and female children aged 5 to 15 years. Then, the children evaluated the flavor and taste of the beverages and evaluated the preference on a scale of 1 to 10. For relative evaluation, the low-dose Artemisia dubia Wall. extract prepared in Preparation Example 1 of the present disclosure was set at score 5, and the evaluation results were expressed as values relative to score 5. The results were summarized and averaged, and the average values are shown in Table 8 below. In Table 8 below, the higher the score, the better the preference.

TABLE 8

| | M1 | M2 | M3 | M4 | M5 | M6 | M7 |
|---|---|---|---|---|---|---|---|
| Taste | 5 | 5.2 | 5.5 | 6 | 5.8 | 5.3 | 6.15 |
| Flavor | 5 | 5.3 | 5.7 | 6.3 | 6 | 5.5 | 6.2 |
| Overall | 5 | 5.25 | 5.6 | 6.15 | 5.9 | 5.4 | 6 |
| preference | | | | | | | |

(unit: score)

Referring to Table 8 above, it was confirmed that M2 to M6 further containing the Thymus vulgaris L. extract exhibited a slightly increased preference compared to M1, and specifically, the mixtures M3 to M5 exhibited an excellent preference. In addition, it was confirmed that the mixture M4 among the mixtures M2 to M6 as well as M7 exhibited the highest preference. It was confirmed that, although M7 further includes γ-oryzanol, the additional inclusion of γ-oryzanol did not affect the preference.

Although the preferred embodiments of the present disclosure have been described in detail above, the scope of the present disclosure is not limited thereto, and various modifications and improvements made by those skilled in the art using the basic concept of the present disclosure defined in the following claims also fall within the scope of the present disclosure.

What is claimed is:

1. A composition for promoting height growth comprising an Artemisia dubia Wall. extract as an active ingredient.

2. The composition according to claim 1, wherein the extract is obtained by extraction with an extraction solvent selected from the group consisting of water, a C₁-C₆ lower alcohol, and a mixture thereof.

3. The composition according to claim 1, wherein the composition exhibits an effect of increasing expression of a growth factor.

4. The composition according to claim 3, wherein the growth factor is IGF-1 gene.

5. The composition according to claim 1, further comprising a Thymus vulgaris L. extract.

6. The composition according to claim 1, wherein the composition exhibits an effect of increasing body weight, increasing tibial growth plate, or increasing femoral growth plate.

7. A food composition for promoting height growth, comprising the composition for promoting height growth according to claim 1.

8. A pharmaceutical composition for promoting height growth, comprising the composition for promoting height growth according to claim 1.
